(19) United States
(12) Patent Application Publication
(10) Pub. No.: US 2021/0186314 A1
(43) Pub. Date: Jun. 24, 2021

(54) DUAL ENDOSCOPE DEVICE AND METHODS OF NAVIGATION THEREFOR

(71) Applicant: Canon U.S.A., Inc., Melville, NY (US)

(72) Inventors: Charles George Hwang, Wellesley, MA (US); Alexander Altshuler, Cambridge, MA (US); James Hastings Houskeeper, Mendon, MA (US)

(21) Appl. No.: 17/111,001

(22) Filed: Dec. 3, 2020

Related U.S. Application Data
(60) Provisional application No. 62/952,770, filed on Dec. 23, 2019.

(51) Int. Cl. A61B 1/012; A61B 1/00; A61B 1/04; A61B 1/018

(52) U.S. Cl. CPC A61B 1/0125; A61B 1/018; A61B 1/04; A61B 1/00045

(57) ABSTRACT

A dual endoscope system, comprising: a main endoscope having a first tubular shaft extending between proximal and distal ends; a secondary endoscope having a second tubular shaft with a distal portion bent with respect to the main endoscope; and a display device to display an image received from the main endoscope and/or the secondary endoscope. The main and secondary endoscopes are joined together along a length of the first and second tubular shafts and are configured to be simultaneously inserted into a lumen. The main endoscope is arranged to acquire a first image of a field of view inside the lumen, and the secondary endoscope is arranged to acquire a second image at an angle to the field of view. The display device displays the first image acquired by main endoscope and a graphic object depicting position and/or orientation of the secondary endoscope with respect to the main endoscope.

### CROSS-REFERENCE TO RELATED APPLICATIONS

The present application claims priority to U.S. provisional application 62/952,770, filed Dec. 23, 2019, the disclosure of which is incorporated by reference herein in its entirety.

### BACKGROUND INFORMATION

### Field of Disclosure

The present disclosure relates to medical devices. More particularly, the disclosure exemplifies various embodiments of a dual endoscope system and methods of operation therefor.

### Description of Related Art

Medical imaging probes, such as endoscopes and catheters, can be inserted through natural orifices or small surgical incisions of a patient's body to provide detailed images from inside the patient's body while being minimally invasive to the patient's comfort. An endoscope is a medical device comprising a tubular conduit with one or more longitudinal channels through which a bodily lumen can be imaged, examined, and/or treated. Endoscopes which include a camera mounted on the distal end of the tube can provide visual access to difficult to reach areas while a medical professional navigates the endoscope into a body cavity looking for abnormalities. These difficult to reach areas are, at times, in sensitive areas where navigation errors can cause harm to the patient. The likelihood of navigation error is small when it is a rigid zero-degree endoscope, which looks straight ahead in a forward direction, and the user can observe the image through a video monitor. However, as the orientation of the scope deviates from zero degrees in order to provide lateral views, or when the endoscope is flexible and it travels through tortuous paths, the presentation of images on a video monitor, and the navigation based on such images becomes more and more difficult.

Previous attempts to address the above-described issues include image guided navigation, for example, as described in U.S. Pat. Nos. 5,638,819 and 8,000,890. However, image guided navigation generally relies on extensive three-dimensional (3D) computer enhancement and reconstruction of tomogram images taken prior to the actual navigation procedure. However, no amount of computer enhancement and reconstruction of tomogram images taken at a past point in time could accurately represent the patient's anatomy during real time endoscope navigation. More specifically, although modern computers can perform complex 3D analysis of previously acquired tomogram images in near real time, the actual instrument positioning and navigation could still be hampered by changes in the patient's anatomy or patient's movement. That is, image navigation systems which are based on previously acquired tomograms merely track actions already taken by the endoscope user, but fail to adequately inform the user of what actions are necessary to take in order to safely guide an instrument along a specific trajectory without causing damage to a patient.

Other attempts to improve endoscope navigation towards difficult to reach areas includes the provision of dual-view endoscopes which include dual viewing ports one for forward viewing and one for lateral viewing, for example, as described in U.S. Pat. No. 4,846,154. U.S. Pat. Nos. 6,554,767 and 8,182,422 disclose an endoscope device and a component attachable-to and detachable-from the endoscope distal end to provide an existing endoscope with an auxiliary imaging device. Multiple endoscopes are occasionally used in combination. By way of example, a so-called mother endoscope may be used with a so-called daughter or baby endoscope. By way of example, the daughter or baby scope may be used to view areas beyond the reach of the mother endoscope. U.S. Pat. No. 4,979,496 and patent application publication US 2010/0228086 disclose a primary (mother) endoscope into which a secondary (daughter) endoscope is inserted through the working channel of the mother endoscope. In these documents, the daughter endoscope could be used to explore and treat areas lateral or tangential to the mother endoscope. However, mother-daughter endoscope systems generally require two operators (one for each endoscope), and the mother endoscope does not provide a direct view of an insertion path for the daughter endoscope.

Therefore, while there are a variety of endoscopes with front and side-viewing capabilities, endoscopes with attachable auxiliary cameras, and multi-channel endoscopes which can provide improved navigation, these endoscopes are still limited by certain disadvantages. Some of these disadvantages include, but are not limited to, not visualizing insert tools, not indicating the position of inserted tools, or even not having the capability of inserting bent or bendable tools.

Spectrally encoded endoscopy (SEE) probes are submillimeter (miniaturized) imaging probes which employ a few or single optical fibers with a miniature diffraction grating at the distal end of the fiber to image the inside of a bodily lumen. An example of a miniaturized SEE probe is described by Tearney et al., in “Spectrally encoded miniature endoscopy”, published in Opt. Lett. 27: 412-414 (2002). SEE probes can be configured for forward-view imaging or for side-view imaging. In either case, broadband light is delivered by the optical fiber or fibers from a light source to the distal end of the probe and focused by a miniature lens. A diffraction grating, which is positioned after the miniature lens, disperses the broadband light into multiple beams with different wavelengths (colors) to generate a spectrally resolved line of light on the imaging plane. Each line illuminates a sample (e.g., tissue) in a different direction from the end of the probe, and thus encodes light reflected from the sample in a given transverse coordinate by wavelength. A line image of the sample is acquired by digitally analyzing the spectral frequency of light reflected from the tissue and returned by the probe. A two-dimensional (2D) image is formed by slowly scanning the spectrally encoded line on the sample along another transverse coordinate (orthogonal to the first transverse coordinate). The other transverse coordinate, which is typically perpendicular to the spectrally-encoded coordinate, is scanned by rotating the SEE probe with a small motor that is typically located in the endoscope handle outside of the patient.

Miniaturized SEE probes have the potential to more easily navigate and reach hard-to-reach imaging areas within a bodily lumen of a patient. For example, SEE probes can be used to obtain images from inside the maxillary sinus by inserting the endoscope through the natural ostium of a patient. To access the maxillary sinus, by inserting a thin endoscope through the natural ostium, the endoscope should be flexible and/or should have a predefined curved shape. In such cases, endoscope users have to rotate and/or bend the endoscope guide to advance from the entry point (the nasal passage) through a tortuous path (the natural ostium) to reach the target location (maxillary sinus) while observing a live image in a video monitor. A similar issue arises when navigating an endoscope or other imaging probe along other tortuous biological paths, such as navigating a patient's airway going from the trachea through the carina and into the lungs. In this case, to have a more intuitive procedure, endoscope users want the endoscope image orientation to be the same as the patient's orientation so that the user will not lose track of where the endoscope tip is (position) and where it is looking (orientation) while the endoscope advances through the tortuous path towards the specific target location.

However, when the SEE endoscope or other imaging probe is in a tortuous path and needs to access a specific location as the maxillary sinus or lungs described above, and the movement of the endoscope is limited by the geometry of the endoscope and/or the anatomy of the lumen, users cannot intuitively navigate towards the desired specific location. Therefore, there remains a need for an endoscope device which can allow a user to easily navigate through tortuous paths without causing any detriment to the patient's sensitive areas.

### SUMMARY OF EXEMPLARY EMBODIMENTS

According to at least one embodiment of the present disclosure, there is provided an endoscope system, comprising: a main endoscope having a first tubular shaft which is rigid and substantially straight extending from a proximal end to a distal end; a secondary endoscope having a second tubular shaft which has a straight portion and a bent portion at the distal end thereof; and a display device configured to display an image received from the main endoscope and/or the secondary endoscope, wherein the main endoscope and the secondary endoscope are joined together along a length of the first and second tubular shafts and are configured to be simultaneously inserted into a lumen, wherein the main endoscope is arranged to acquire a first image of a field of view from inside the lumen, and the secondary endoscope is arranged to acquire a second image of an area which is tangential or lateral to the field of view, and wherein the display device displays the first image acquired by main endoscope and a graphic object depicting a position and orientation of the secondary endoscope with respect to the main endoscope.

According on an aspect of the present disclosure, it is further provided an endoscope system for performing a medical procedure, comprising: a first endoscope probe having opposite proximal and distal ends, wherein at least a distal portion of the first endoscope probe is rigid and substantially straight and configured to be inserted into a bodily lumen for forward-view imaging; and a second endoscope probe having opposite proximal and distal ends and configured to be inserted into the bodily lumen for side-view imaging, wherein at least the distal end of the second endoscope probe is bent at an angle with respect to a proximal portion thereof. The first endoscope probe and the second endoscope probe are joined together substantially parallel to each other such that the first endoscope probe is arranged to take forward-view images from a field of view that includes the distal end of the second endoscope probe when the second endoscope probe is navigated through the bodily lumen.

These and other objects, features, and advantages of the present disclosure will become apparent upon reading the following detailed description of exemplary embodiments of the present disclosure, when taken in conjunction with the appended drawings, and provided claims.

### BRIEF DESCRIPTION OF DRAWINGS

Further objects, features and advantages of the present disclosure will become apparent from the following detailed description when taken in conjunction with the accompanying figures showing illustrative embodiments of the present disclosure.

FIG. 1 is a diagram showing a first embodiment of a dual-scope endoscope system 100 which includes a main endoscope 150, a secondary endoscope 130, a console 110, and a display 115.

FIG. 2 illustrates an exemplary block diagram of constituent parts of the console 110.

FIG. 3A and FIG. 3B show detailed schematics of an example of the secondary endoscope 130.

FIG. 4A illustrates an example of an arrangement where the main endoscope 150 is attached to the secondary endoscope 160 via a mechanical joint 160. FIG. 4B shows an example of a video image 401 obtained from the main endoscope 150 and displayed on a screen of display 115 together with a graphic object 405 which indicates the position and orientation of the tip of the secondary endoscope 130 with respect to field-of-view of the main endoscope 150.

FIG. 5A shows another example of an arrangement where the main endoscope 150 is attached to the secondary endoscope 160 via a mechanical joint 160. FIG. 5B shows an example of a video image 501 obtained from the main endoscope 150 and displayed on a screen of display 115 together with a graphic object 505 which indicates the position and orientation of the tip of the secondary endoscope 130 with respect to field-of-view of the main endoscope 150.

FIG. 6A shows an embodiment where the secondary endoscope (SEE scope) 130 can be temporarily attached to the main endoscope 150 via pressure fitting cylindrical clamps 161a and 161b. FIG. 6B shows a display 115 with video image 601 together with a graphic object 605 which indicates the position and orientation of the tip of the secondary endoscope 130 with respect to field-of-view of the main endoscope 150.

FIG. 7 shows an embodiment where the secondary endoscope (SEE scope) 130 can be temporarily attached to the main endoscope 150 via a sleeve or cylindrical tube 165.

FIG. 8 shows an embodiment where the secondary endoscope (SEE scope) 130 can be temporarily attached to the main endoscope 150 via a guide rail 162a and an engaging member 162b.

FIG. 9 illustrates an exemplary embodiment of the handle 120 configured to operate one or both of the main endoscope 150 and the secondary endoscope 130.

FIG. 10A illustrates an example of the endoscope system 100 arranged to be used in navigation or insertion mode. FIG. 10B shows an example of the endoscope system 100 arranged to be used in a procedure or imaging mode.

FIG. 11 illustrates a flowchart of a tracking procedure for monitoring in real time the relative position of the secondary endoscope with respect to the main endoscope.

### DETAILED DESCRIPTION OF EXEMPLARY EMBODIMENTS

The exemplary embodiments disclosed herein are based on an objective of providing a first endoscope probe and the second endoscope probe joined together substantially parallel to each other such that the first endoscope probe is arranged to take forward-view images from a field of view that includes the distal end of the second endoscope probe when the second endoscope probe is navigated through a bodily lumen. The second endoscope probe is preferably a fiber-optic-based imaging probe that can be fabricated easily, at low cost, and can maintain the ability to provide high quality images. As used herein, imaging probes and optical elements thereof include miniaturized components having physical dimensions of 1.5 millimeters (mm) or less in diameter.

Throughout the figures, the same reference numerals and characters, unless otherwise stated, are used to denote like features, elements, components or portions of the illustrated embodiments. In addition, while the subject disclosure is described in detail with reference to the enclosed figures, it is done so in connection with illustrative exemplary embodiments. It is intended that changes and modifications can be made to the described exemplary embodiments without departing from the true scope and spirit of the subject disclosure as defined by the appended claims. Although the drawings represent some possible configurations and approaches, the drawings are not necessarily to scale and certain features may be exaggerated, removed, or partially sectioned to better illustrate and explain certain aspects of the present disclosure. The descriptions set forth herein are not intended to be exhaustive or otherwise limit or restrict the claims to the precise forms and configurations shown in the drawings and disclosed in the following detailed description.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached”, “coupled” or the like to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown in one embodiment can apply to other embodiments. It will also be appreciated by those of skill in the art that references to a structure or feature that is disposed “adjacent” to another feature may have portions that overlap or underlie the adjacent feature.

The terms first, second, third, etc. may be used herein to describe various elements, components, regions, parts and/or sections. It should be understood that these elements, components, regions, parts and/or sections are not limited by these terms of designation. These terms of designation have been used only to distinguish one element, component, region, part, or section from another region, part, or section. Thus, a first element, component, region, part, or section discussed below could be termed a second element, component, region, part, or section merely for purposes of distinction but without limitation and without departing from structural or functional meaning.

As used herein, the singular forms “a”, “an”, and “the” are intended to include the plural forms as well, unless the context clearly indicates otherwise. It should be further understood that the terms “includes” and/or “including”, “comprises” and/or “comprising”, “consists” and/or “consisting” when used in the present specification and claims, specify the presence of stated features, integers, steps, operations, elements, and/or components, but do not preclude the presence or addition of one or more other features, integers, steps, operations, elements, components, and/or groups thereof not explicitly stated. Further, in the present disclosure, the transitional phrase “consisting of” excludes any element, step, or component not specified in the claim. It is further noted that some claims or some features of a claim may be drafted to exclude any optional element; such claims may use exclusive terminology as “solely,” “only” and the like in connection with the recitation of claim elements, or it may use of a “negative” limitation.

The term “about” or “approximately” as used herein means, for example, within 10%, within 5%, or less. In some embodiments, the term “about” may mean within measurement error. In this regard, where described or claimed, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range, if recited herein, is intended to include all sub-ranges subsumed therein. As used herein, the term “substantially” is meant to allow for deviations from the descriptor that do not negatively affect the intended purpose. For example, deviations that are from limitations in measurements, differences within manufacture tolerance, or variations of less than 5% can be considered within the scope of substantially the same. The specified descriptor can be an absolute value (e.g. substantially spherical, substantially perpendicular, substantially concentric, etc.) or a relative term (e.g. substantially similar, substantially the same, etc.).

The present disclosure generally relates to medical devices, and it exemplifies embodiments of an optical probe which may be applicable to a spectroscopic apparatus (e.g., an endoscope), an optical coherence tomographic (OCT) apparatus, or a combination of such apparatuses (e.g., a multi-modality optical probe). The embodiments of the optical probe and portions thereof are described in terms of their state in a three-dimensional space. As used herein, the term “position” refers to the location of an object or a portion of an object in a three-dimensional space (e.g., three degrees of translational freedom along Cartesian X, Y, Z coordinates); the term “orientation” refers to the rotational placement of an object or a portion of an object (three degrees of rotational freedom—e.g., roll, pitch, and yaw); the term “posture” refers to the position of an object or a portion of an object in at least one degree of translational freedom and to the orientation of that object or portion of object in at least one degree of rotational freedom (up to six total degrees of freedom); the term “shape” refers to a set of posture, positions, and/or orientations measured along the elongated body of the object. As it is known in the field of medical devices, the terms “proximal” and “distal” are used with reference to the manipulation of an end of an instrument extending from the user to a surgical or diagnostic site. In this regard, the term “proximal” refers to the portion of the instrument closer to the user, and the term “distal” refers to the portion of the instrument further away from the user and closer to a surgical or diagnostic site.

As used herein the term “catheter” generally refers to a flexible and thin tubular instrument made of medical grade material designed to be inserted through a narrow opening into a bodily lumen (e.g., a vessel) to perform a broad range of medical functions. The more specific term “optical catheter” refers to a medical instrument comprising an elongated bundle of one or more flexible light conducting fibers disposed inside a protective sheath made of medical grade material and having an optical imaging function. A particular example of an optical catheter is a fiber optic catheter which comprises a sheath, a coil, a protector and an optical probe. In some applications a catheter may include a “guide catheter” which functions similarly to a sheath.

As used herein the term “endoscope” refers to a rigid or flexible medical instrument which uses light guided by an optical probe to look inside a body cavity or organ. A medical procedure, in which an endoscope is inserted through a natural opening, is called an endoscopy. Specialized endoscopes are generally named for how or where the endoscope is intended to be used, such as the bronchoscope (mouth), sigmoidoscope (rectum), cystoscope (bladder), nephroscope (kidney), bronchoscope (bronchi), laryngoscope (larynx), otoscope (ear), arthroscope (joint), laparoscope (abdomen), and gastrointestinal endoscopes.

In the present disclosure, the terms “optical fiber”, “fiber optic”, or simply “fiber” refers to an elongated, flexible, light conducting conduit capable of conducting light from one end to another end due to the effect known as total internal reflection. The terms “light guiding component” or “waveguide” may also refer to, or may have the functionality of, an optical fiber. The term “fiber” may refer to one or more light conducting fibers. An optical fiber has a generally transparent, homogenous core, through which the light is guided, and the core is surrounded by a homogenous cladding. The refraction index of the core is larger than the refraction index of the cladding. Depending on design choice some fibers can have multiple claddings surrounding the core.

As outlined above, there is a need for an endoscope device which can provide lateral views and still allow a user to easily navigate through tortuous paths without causing any detriment to the patient's sensitive areas. A solution outlined in this disclosure is to incorporate a main endoscope (mother endoscope) in conjunction with a secondary (daughter) endoscope in order to enable a user to maintain precise tracking and orientation of the daughter endoscope during navigation.

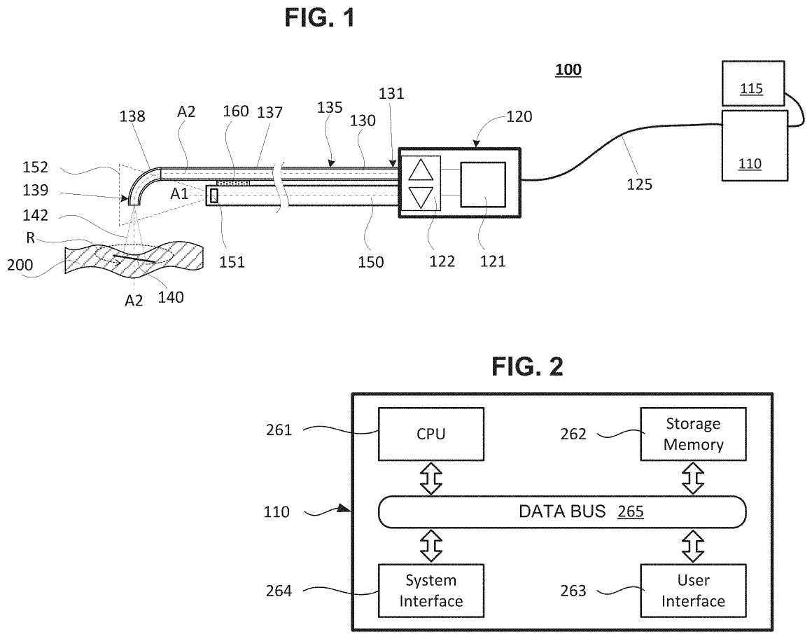

### <FIG. 1>

FIG. 1 shows a first embodiment of a dual-scope endoscope system 100 including a main endoscope 150 and a secondary endoscope 130. The main endoscope 150 is not limited to any particular type of endoscope, but the secondary endoscope 130 is preferably a miniaturized endoscope (i.e., smaller than the main endoscope), such as, an SEE endoscope device, for example.

The endoscope system 100 includes a console 110, a display 115, a handle 120, a main endoscope 150, and a secondary endoscope 130. The console 110 and the handle 120 are operably connected to each other by a cable bundle 125. The display 115 is an image display device, such as an LCD, LED, OLED monitor, which shows a live view image (video image) acquired by the main endoscope 150, and a processed endoscopic image acquired by the secondary endoscope 130. According to one embodiment, the main endoscope 150 and the secondary endoscope 130 are removably attached to each other by a mechanical joint 160.

The main endoscope 150 may be implemented as any suitable device for use in a medical procedure, and which is configured to obtain a live image (i.e., a video image) within a field of view 152 of a site where a medical procedure is to be performed. The main endoscope 150 is not particularly limited to any specific implementation, as long as it is a suitable device for use in a medical procedure, and is configured to obtain a live image (a video image) of a lumen. To that end, the main endoscope 150 is shaped as a substantially tubular shaft extending along a longitudinal axis A1. The main endoscope 150 may include at least an imaging device 151, such as imaging chip (e.g., a CMOS or CCD sensor) disposed at the distal end of the tubular shaft, and may include additional hardware necessary for image acquisition and for navigating the endoscope through a lumen. For example, the main endoscope 150 may include, in addition to the imaging device 151, a guide wire, a catheter, a biopsy or ablation needle, or other similar devices. The main endoscope 150 may also include, in addition to the imaging device 151, one or more working channels for the manipulation of tools (e.g., forceps or tweezers) and for delivery or extraction of fluids such as blood or gas.

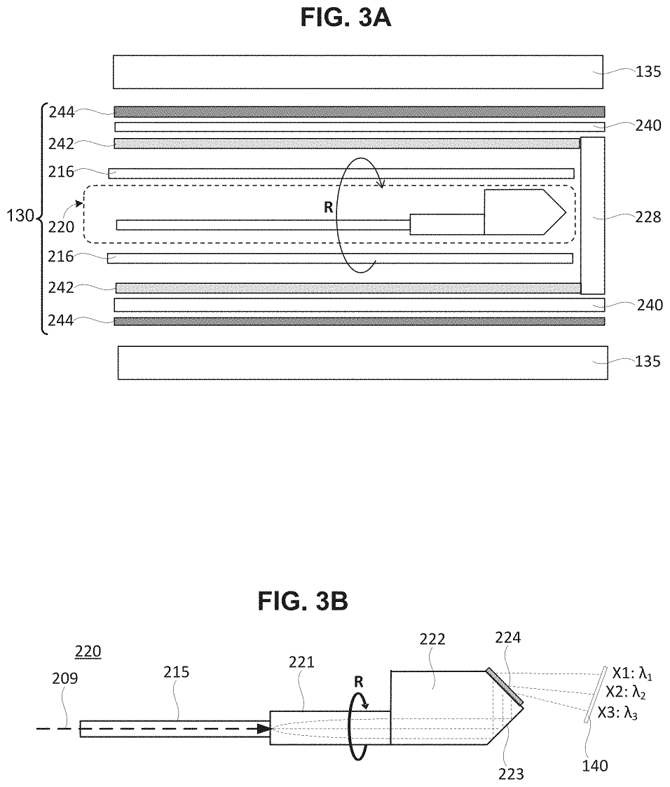

The secondary endoscope 130 is enclosed in an endoscope guide 135 which is independent from (not part of) the main endoscope 150. The endoscope guide 135 is a tubular shaft having a longitudinal axis A2 and extending from a proximal end 131 to a distal end 139. According to at least one embodiment, the endoscope guide 135 may include a distal section 138 and a proximal section 137. The proximal section 137 is substantially strain and linear, while the distal section 139 is bent, bendable, or steerable. The proximal section 137 is substantially parallel to the shaft of the main endoscope 150. The distal section can be bent at an angle in a range from about 25 to 90 degrees with respect to shaft of the main endoscope 150. The endoscope guide 135 contains inside the tubular shaft thereof, among other things, the secondary endoscope 130 which in turn includes endoscope optics also referred to as an optical probe. Endoscope optics includes at least illumination optics and detection optics, as described more in detail with respect to FIG. 3A and FIG. 3B. In at least some embodiments, the secondary endoscope may also include certain end effectors.

In an embodiment where the secondary endoscope 130 is an SEE endoscope, the illumination optics emits a illumination light within a field of view 142, such that a spectrally-encoded illumination light 140 reaches a sample 200 which is tangential to the FOV 152 of the main endoscope 150. In an SEE endoscope, the detection optics collects light reflected and/or scattered by the sample 200 (e.g., an inner wall of a bodily lumen or an area adjacent or lateral to the lumen). The sample 200 can be a hard-to-reach area in a bodily lumen of a patient. For example, in nasal endoscopy, the secondary endoscope 130 may include an SEE probe inside an endoscope guide 135 used to obtain images from inside the maxillary sinus by inserting the secondary endoscope through the natural ostium of a patient.

The endoscope guide 135 can be a rigid and curved tubular shaft with a predetermined angle of orientation which bends towards (or away from) the main endoscope 150. In some embodiments, the endoscope guide 135 of the secondary endoscope 130 can be at least partially flexible and configured to be actively bent (e.g., by kinematic actuation) with respect to the main endoscope 150. The handle 120 is configured to enable a user to manually operate the main endoscope 150 and/or the secondary endoscope 130. The handle 120 may include a controller circuit 121 and an interface unit 122 which are configured to indicate or select which endoscope among the main endoscope 150 and the secondary endoscope 130 should be controlled during a procedure.

For an exemplary nasal endoscopy procedure, the main endoscope 150 may be a zero-degree (straight) nasal endoscope which allows for a straight view into the patient's nose through the nostril to examine the nasal passages. The secondary endoscope 130 may be a flexible or pre-curved endoscope (e.g., pre-shaped at 30, 45, 70 or 90 degrees of angled curvature) to allow for deeper “around-the-corner” views into the patient's difficult-to-reach areas, such as sinus cavities or the maxillary sinus. The use of the two endoscopes simultaneously can provide maximum visualization of the patient's sensitive areas to make diagnoses and/or perform procedures with high accuracy and enhanced patient safety.

Endoscopic data from the main endoscope 150 may be captured according to one or more of various endoscopic or catheter imaging modalities, including video endoscopy (through a videoscope), spectroscopy, fluoroscopy, optical coherence tomography (OCT), e.g., using an OCT catheter, or other similar endoscopic modalities. In some embodiments, the main endoscope 150 may include a working channel for one or more medical instruments and means for providing a forward view image of the lumen; the forward view of the lumen can be used as a live view for navigation, or can be stored in the system for correlation with the imaging of the secondary endoscope 130. In some embodiments, the secondary endoscope 130 may be permanently attached to an outer surface of the main endoscope 150. In other embodiments, the mother or main endoscope 150 may function as a primary modality such as an OCT catheter, while the daughter of secondary endoscope 130 may be temporarily attached to the side of the main endoscope 150 to aid the navigation of the main endoscope. Alternatively, main endoscope 150 aids in the navigation of the secondary endoscope 130. In either case, image data from the two endoscopes is preferably recorded and processed separately by the console 110, but the images can be viewed together or separately in the display 115, as desired by the user.

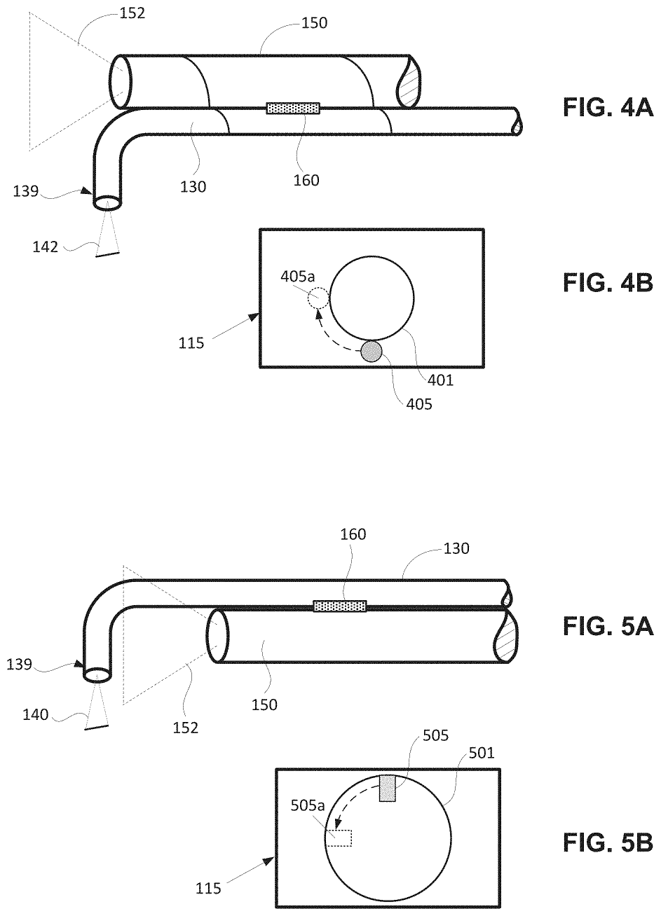

As shown in FIG. 1, the secondary endoscope 130 is arranged in close proximity, and substantially parallel, to the main endoscope 150 such that the distal end 139 (the tip) of the secondary endoscope 130 is within the field of view 152 of the main endoscope 150. In this manner, the user can observe the tip of the secondary endoscope in the field of view shown on the mother endoscope monitor, as further explained with reference to FIG. 4B and FIG. 5B discussed below. To facilitate assembling, reduce space, and improver procedure accuracy, the first axis A1 of the main endoscope 150 and the second axis A2 of the secondary endoscope 130 are parallel to each other at least a portion of their length thereof.

### <FIG. 2>

As mentioned above, the endoscope system 100 includes a console 110 and a handle 120 which are in operable communication with each other to control the operations of one or both of the main endoscope 150 and the secondary endoscope 130. FIG. 2 illustrates an exemplary block diagram of constituent components of the console 110. Console 110 may be implemented by, for example, a general purpose computer specifically programmed with algorithms to execute endoscope navigation and image orientation control, for example, as described with reference to FIGS. 4B and 5B. The console 110 includes or is operably attached to the display 115 for displaying the images acquired with endoscope system 100. To that end, console 110 includes a central processing unit (CPU) 261, a storage memory (ROM/RAM) 262, a user input/output (I/O) interface 263, and a system interface 264 which are all interconnected via a data bus 265. The console 110 can programmed to issue a command that can be transmitted to the various parts of the imaging system 100 upon receiving a user input via the user interface 263. An input device, such as key board, a mouse, and/or a touch panel screen in the display 115 can be provided as part of the user interface 263.

The CPU 261 may be configured to read and perform computer-executable instructions stored in the storage memory 262. The computer-executable instructions may include program code for the performance of the methods, measurements, and/or calculations of the system 100, as described herein. For example, CPU 261 may receive signals from handle 120 corresponding to a selection or operation of the main endoscope 150 or the secondary endoscope 130 to obtain images from a bodily lumen sample 200.

The system interface 264 provides an electronic interface for the various components connected to or provided in the console 110. For example, the system interface 264 provides an electronic interface for one or more a light source (not shown) which emits broadband light to the second endoscope 130, a detector or spectrometer (not shown), the cable bundle 125, and the display 115. The system interface 264 includes electronics necessary to receive electrical signals corresponding to images acquired by the main endoscope 150 and the secondary endoscope 130, and to output a video signal out to the display 115.

The console 110 may contain, in addition to a CPU 261, for example, one or more of a field-programmable gate array (FPGA), a digital signal processor (DSP), an application specific integrated circuit (ASIC), a graphic processing unit (GPU), a system on chip (SoC) or combinations thereof, which perform some or the entire image processing and signaling of the endoscope system 100.

### <FIG. 3A and FIG. 3B>

FIG. 3A and FIG. 3B show detailed schematics of an example of the secondary endoscope 130. As shown in FIG. 3A, the secondary endoscope 130 is enclosed inside the endoscope guide 135. The secondary endoscope 130 includes an outer sheath 244, an inner sheath 242, a drive cable 216, and a probe 220 arranged enclosed in the inner diameter of drive cable 216. The drive cable 216 is rotated together with the probe 220 inside of the inner sheath 242. The inner sheath 242 is surrounded by detection optics 240 which can be a ring of detection waveguides 245 (e.g., a ring of optical fibers or a fiber bundle). The inner sheath 242 supports a transparent window 228 at the distal end of the endoscope 130. The transparent window 228 protects the illumination optics of probe 220 from the outside environment surrounding the endoscope guide. Concentrically surrounding the detection optics 240 there is provided the outer sheath 244 to protect the ring of optical fibers or fiber bundle which constitutes the detection optics 240. All of the foregoing components are enclosed inside of the inner diameter of endoscope guide 135. As described above, the endoscope guide 135 can be rigid or flexible, and it is preferably bent at the distal end thereof to facilitate navigation and side-view imaging of specific anatomical features, such as the maxillary sinus. At the proximal end, the endoscope guide 135 is fixedly connected to the endoscope handle 120. In an imaging operation, the endoscope guide 135 is not mechanically rotated, but the user operates the endoscope guide 135 to pitch, roll, and change its direction of view, by manipulating the endoscope handle 120.

As shown in FIG. 3B, the illumination optics probe 220 includes an illumination waveguide 215 which can be a single mode or multi-mode fiber (a light guiding element), a focusing element 221 which can be a graded index (GRIN) lens or a ball lens, a spacer 222, and a diffractive element 224. The spacer 222 can be a transparent component having at least two surfaces configured to guide illumination light 209 provided through the illumination waveguide 215 and the focusing element 221. Specifically, the spacer 222 includes a first surface which is a reflective surface 223 and a second surface which includes a grating surface containing the diffractive element 224. The spacer 222 can be made of transparent plastic, e.g., by injection molding, or can be made of glass, e.g., by glass compression molding, or it can be a piece of coreless optical fiber. The reflective surface 223 can be made by polishing a part of the spacer to satisfy total internal reflection (TIR) conditions, or it can be a mirror-coated surface. The second surface containing the diffractive element 224 can be made by applying UV-curable resin on the second surface of the spacer 222 and stamping a master grating on the resin, by a nanoimprint technique, e.g., as described in U.S. patent Ser. No. 10/261,223 which is incorporated by reference herein in its entirety. The illumination light 209 from the illumination waveguide 215 is slightly focused by the focusing element 221 and reflected by the reflective surface 223, and thereafter diffracted by the diffractive element 224 so that a spectrally-encoded illumination line 140 is formed over the sample 200, at a working distance from the probe's distal end.

### <FIG. 4A-5B: Navigation and Tracking>

Turning now to FIG. 4A through 5B an example of navigation and tracking of the dual endoscope is described. FIG. 4A shows an example embodiment where the main endoscope 150 and the secondary endoscope 160 are attached to each other via a mechanical joint 160. As shown in FIG. 4A, at least a portion of the main endoscope 150 at the distal end thereof is rigid and substantially straight (i.e., not curved or bent). On the other hand, at least a portion of the secondary endoscope 130 at the distal end thereof is bent at a predetermined angle, or is flexible so as to be bent at an arbitrary angle, with respect to the main endoscope 150. In the arrangement shown in FIG. 4A, the distal portion of the secondary endoscope 130 is bent away from the shaft of the main endoscope 150. This particular arrangement can be advantageous in certain applications, such as for imaging the maxillary sinus by inserting the secondary endoscope via the natural ostium of a patient's ostiomeatal complex (OMC). OMC is a common channel that links the frontal sinus, anterior ethmoid air cells and the maxillary sinus to the middle meatus, allowing airflow and mucociliary drainage. The distal end of the secondary endoscope 130 can be bent a priori or can be actively controlled to bend, for example, by about 30, 45 or 70 degrees, which are typical inclinations for nasal endoscopes.

As it will be understood by persons skilled in the art, endoscope navigation through the OCM channel possesses significant challenges in that the OCM channel represents a highly tortuous path, where it is important that the tip of the endoscope is visible at all times and no pressure whatsoever is exerted on the lateral nasal wall to prevent accidental discomfort or injury to the patient. To that end, according to the present disclosure, it is advantageous to use a highly flexible or pre-angled ultrathin secondary endoscope together with a conventional endoscope. The secondary endoscope 130 can have a pre-set shape, or can be steerable (e.g., by kinematic action) from the handle 120. In the case where the secondary endoscope 130 is bent away from the main endoscope 150 and the distal end of the secondary endoscope is not within the field of view 152 of the main endoscope (e.g., as illustrated in FIG. 4A), the mechanical joint 160 can be used as a reference point to track the orientation of the secondary endoscope 130 with respect to the main endoscope 150 during insertion into a lumen.

To track the orientation of the secondary endoscope 130 with respect to the main endoscope 150, the console 110 receives a video image from the main endoscope 150 and displays the video image on a screen of a display 115. FIG. 4B shows an example of a video image 401 obtained from the main endoscope 150 and displayed on a screen of display 115. To track the orientation of the secondary endoscope 130, the display 115 also shows a pointer 405 (a graphic object) which corresponds to the position of mechanical joint 160 with respect to the main endoscope 150. In this manner, during a procedure, when the endoscope operator uses the main endoscope 150 to advance through a lumen, the pointer 405 will be shown at a fixed position with respect to video image 401. And, when the operator rotates the main endoscope 150, the pointer 405 shown on the display 115 will move around the edge of video image 401 to show the direction and amount of rotation. In FIG. 4B, the pointer 405 is shown as a temporary pointer 405a indicating a clockwise rotation of the dual endoscope. This will inform the user of the exact position and orientation of the tip of the secondary endoscope 130 with respect to the lumen being imaged. According to the arrangement of FIG. 4A, the main endoscope 150 and the secondary endoscope 130 can rotate locked together as unit. In this case, pointer 405 can be advantageously used to show the endoscope operator the direction in which the tip of the secondary endoscope is pointed to. In other embodiments, however, the main endoscope 150 and the secondary endoscope 130 may rotate independently of each other.

FIG. 5A shows another example where the main endoscope 150 and the secondary endoscope 160 are attached to each other via a mechanical joint 160. As show in FIG. 5A, the distal portion of the secondary endoscope 130 is bent towards the shaft of the main endoscope 150. In this arrangement, the main endoscope 150 is arranged to take forward-view images from a field of view 152 that includes the distal end 139 of the secondary endoscope 130. With this arrangement, the secondary endoscope 130 can be used to obtain side-view images of the bodily lumen or of areas tangential to the lumen, while the main endoscope 150 is used for live view navigation through the lumen.

This particular arrangement can be advantageous in certain applications, such as for imaging the maxillary sinus by inserting the endoscope via the natural ostium and constantly monitoring that the distal end 139 of the secondary endoscope 130 exerts no pressure whatsoever on the lateral nasal wall to prevent accidental injury to the patient.

Similar to the previous example, the console 110 can show a live image in the display 115 to track in real time the position of the main endoscope 150 and the orientation of the secondary endoscope 130 with respect to the main endoscope. To that end, the console 110 receives a video image from the main endoscope 150 and displays the video image on a screen of display 115. FIG. 5B shows an example of a video image 501 as it would be obtained from the main endoscope 150 and displayed on a screen of display 115. To track the orientation of the secondary endoscope 130, the display 115 also shows either a live image of the distal end 139 of secondary endoscope 130 and/or a pointer 505 inside the edge of the image 501. In this manner, during a procedure, the image of the distal end of the secondary endoscope or the pointer 505 shown on the display 115 will move together with the video image 501 and will track the position/orientation of the secondary endoscope with respect to the main endoscope. In FIG. 5B, an initial position (12 o'clock) of the pointer 505 is shown as rotating in a counter clockwise direction to a second position (9 o'clock) as pointer 505a. This will inform the user of the exact position and orientation of the tip of the secondary endoscope 130 with respect to lumen being imaged.

In the foregoing illustrations of FIG. 1, FIG. 4A, and FIG. 5A the secondary scope can be considered to be fixedly attached to the main endoscope 150 in a known position relative to each other. In this case, the mechanical joint 160 can be a permanent attachment, such as a mechanical weld, permanent bond by adhesive, or pressure fitting, a keyway and pin engagement, such that the position and orientation of the secondary endoscope 130 is substantially permanently fixed to the main endoscope 150. In this case, because the position of the secondary endoscope is relatively fixed to the main endoscope 150, an indicator can be displayed at all times on the endoscope monitor showing the relative position between the two endoscopes. In alternative embodiments, the mechanical joint can be a non-permanent joint such that the position and orientation of the secondary endoscope 130 with respect to the main endoscope 150 can be changed.

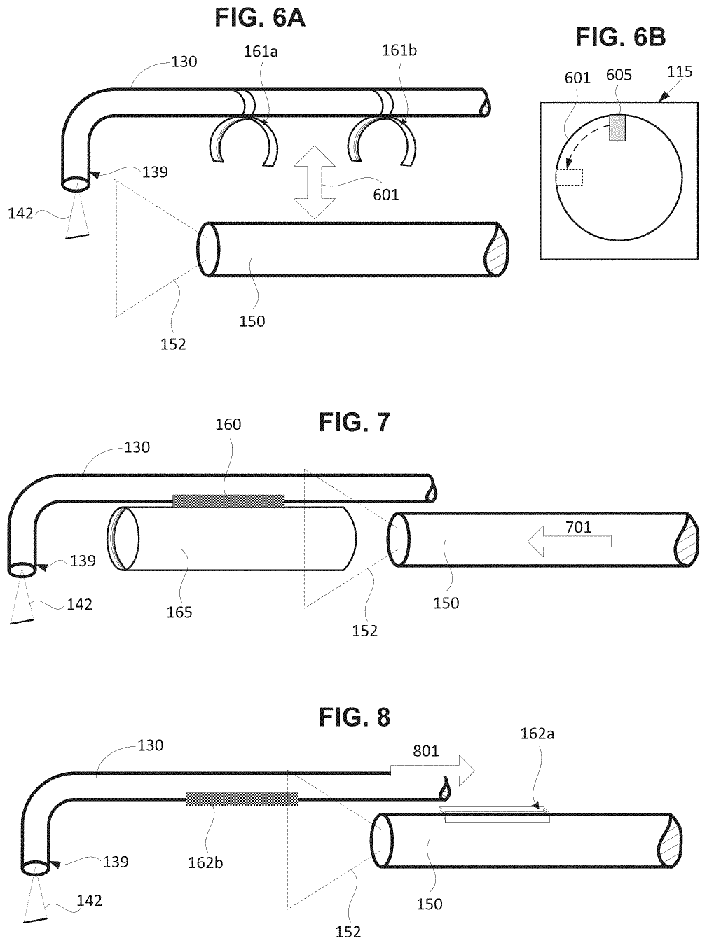

### <FIG. 6A-8: Mechanical Joint>

FIG. 6A, FIG. 7, and FIG. 8 show various alternative embodiments for implementing the mechanical joint 160 to join together the main endoscope 150 to the secondary endoscope 130. FIG. 6A shows an embodiment where the secondary endoscope (e.g., an SEE scope) 130 can be temporarily attached to the main endoscope 150 via pressure-fitted cylindrical clips. In this case, the mechanical joint 160 includes a connection clip assembly comprised of a plurality of pressure fitting cylindrical clamps 161a and 161b which are sized to engage around the outer surface of the main endoscope 150. Naturally, the cylindrical clamps 161a and 161b can be provided on the main endoscope and sized to engage around the outer surface of the secondary endoscope 130. The two endoscopes can be easily attached to and detached from each other by a simple mechanical action 601 of bringing one endoscope towards the other and pressure fitting the clamps 161a and 161b over the outer surface of the tubular shaft of the main endoscope. To avoid longitudinal slippage of one endoscope with respect to the other, the main endoscope 150 may be provided with annular lips, rings, or channels to abut against one or more the cylindrical clamps.

FIG. 6B shows an example of a video image 601 obtained from the main endoscope 150 and displayed on a screen of display 115. In this arrangement, since the distal end 139 of the secondary endoscope 130 is bent towards the field of view of main endoscope 150, the display 115 can show an actual image of the tip of the secondary endoscope 130. Alternatively, the display 155 can show a graphic object 605 such as pointer or circle or other making inside the edge of the image 601 (superposed on part of the video image). This graphic object serves to inform the user of the relative position of the secondary endoscope with respect to the main endoscope. In this manner, during a navigating procedure where the secondary endoscope may rotate with respect to the main endoscope, the graphic object 605 corresponding to the distal end of the secondary endoscope will move along the edge of the video image 601 (as shown by the dashed arrow). In FIG. 6B, an initial position/orientation of the secondary endoscope 130 is shown as the graphic object 605 and a second positon/orientation of the secondary endoscope is shown as a graphic object 605a. This will inform the user of the exact position, orientation and rotational direction of the tip of the secondary endoscope 130 with respect to field-of-view of the main endoscope 150. In other words, the graphic object 605 is a probe tip indicator configured to provide information about position and orientation of the secondary endoscope with respect to the main endoscope.

FIG. 7 shows an embodiment where the secondary endoscope (e.g., an SEE scope) 130 can be temporarily attached to the main endoscope 150 in a known position via a cylindrical sleeve. In this case, the mechanical joint 160 includes a cylindrical tube 165 which is sized to fit the outer diameter of main endoscope 150. The cylindrical tube 165 works as a coupling sleeve which defines a cylindrical opening for receiving therein the main endoscope 150. The two endoscopes can be easily attached and detached from each other by a simple mechanical action 701 of sliding the main endoscope 150 into the cylindrical tube 165. The cylindrical tube 165 can be permanently welded, or otherwise it can be temporarily secured to the secondary endoscope 130. In either case, the mechanical joint 160 serves to maintain the main endoscope 150 in a known position with respect to the secondary endoscope 130.

FIG. 8 shows an embodiment where the secondary endoscope (e.g., an SEE scope) 130 can be temporarily attached to the main endoscope 150 via a rail structure. In this case, the mechanical joint 160 includes a track in the form of a guide rail 162a provided in a first one of the two endoscopes (provided in the main endoscope 150) and an engaging member 162b in the form of a flange or a pillar provided on the second one of the two endoscopes. Endoscope mechanical junctions of this type are described, for example, in US patent application publication US 2004/0230096, the disclosure of which is incorporated by reference herein. With this arrangement, the two endoscopes can be easily attached and detached from each other by a simple mechanical action 801 of bringing one endoscope towards the other and sliding the engaging member 162b into the guide rail 162a. Because the position of the secondary endoscope 130 (SEE scope) is relatively fixed to the main endoscope 150, an indicator (similar to graphic object 605) can be displayed in the endoscope monitor showing the position of the secondary endoscope with respect to the main endoscope.

Any of the mechanical joints shown in FIG. 6, FIG. 7, and FIG. 8 can be made from medical grade plastic material, such as polyethylene, Teflon®, or polypropylene to provide a low coefficient of friction between the members as they slide relative to one another. Alternatively, these mechanical joints can be made of medial grade metal (e.g., stainless steel or nitinol) covered with biocompatible lubricious polymers having a low friction coefficient. Additionally, while the mechanical joint 160 is shown and described as being formed from two or more separate parts, the mechanical joint 160 can be formed as a unitary piece, in particular when the two endoscopes are permanently joined together. Likewise, the mechanical joint 160 can be custom made to join an already existing conventional rigid zero-degrees endoscope with an ultrathin submillimeter flexible endoscope, such as the SEE endoscope shown in FIG. 3A-FIG. 3B. In this case, the mechanical joint 160 can be formed as a unitary piece (e.g., as a cylindrical clamp shown in FIG. 6A) made by an extrusion process or molding process, and thereafter the unitary piece can be joined to either the main endoscope or the secondary endoscope by any suitable attachment method or material. In some embodiments, at least part of the mechanical joint 160 can be made of, or it can include, a radiopaque material, so that the joint 160 can serve as a radiopaque marker in certain image guided procedures. As a further alternative, at least part of the mechanical joint 160 can be made of magnetic material (e.g., by mixing magnetic particles into extrusion polymer material or adding magnets to stainless steel or nitinol metal) covered with biocompatible lubricious additives.

While one mode of operation of the dual endoscope system 100 would be to have the main and secondary scopes move/rotate together as a fixed unit. In some embodiments, as described below, the dual endoscope system 100 can be operated in a manner that the main endoscope and secondary endoscope move/rotate independent from each other even if they are joined prior to insertion into a lumen.

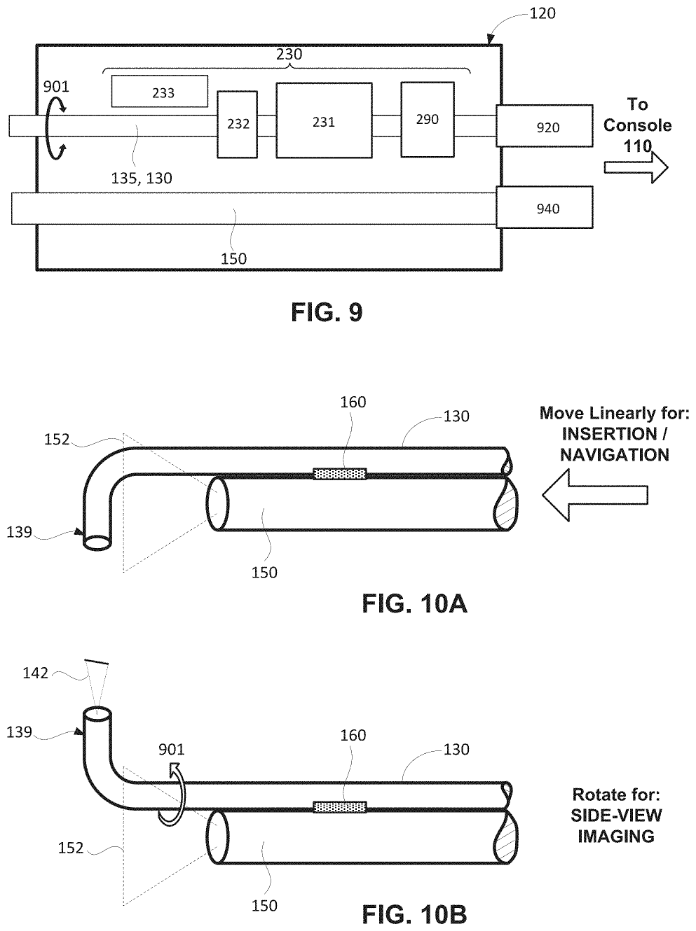

### <FIG. 9-FIG. 10: Exemplary Modes of Operation>

FIG. 9 illustrates an embodiment of the handle 120 configured to connect the main endoscope 150 and the secondary endoscope 130 to the console 110 shown in FIG. 1. The handle 120 may include a first connector 940 and a second connector 920 respectively configured to connect the main endoscope 150 and the secondary endoscope 130 to the console 110. The dual endoscope system 100 can operate in a navigation mode and an imaging mode. A navigation mode refers to a mode of operation of the endoscope system 100 to insert the two endoscopes into a bodily lumen, and linearly advance at least one of the main and secondary endoscopes to a specific location inside the lumen. An imaging mode refers, for example, to a mode of obtaining an endoscopic image with the use of the secondary endoscope after navigating the distal end 139 of secondary endoscope 130 to the desired location. To do that, according to FIG. 9, for example, the handle 120 may include a rotation mechanism 230 which can be controlled by the controller 121 (located in the handle 120) or by the console 110 (as shown in FIG. 1). The rotation mechanism 230 may include a first motor 231, a second motor 290, and a tracking mechanism composed of a rotating target 232 and a sensor 233.

During an imaging operation, the rotation mechanism 230 can use a hollow-shaft motor 231 which can be configured to rotate or oscillate the secondary endoscope 130 inside its endoscope guide 135. A tracking mechanism such as an encoder comprised of the rotating target 232 and sensor 233 can track rotation and orientation of the endoscope 130. However, during a navigation operation (e.g. during insertion towards a desired lumen location), an additional rotation mechanism (e.g., a second hollow-shaft motor 290 or other rotating mechanism) can be configured to also rotate the endoscope guide 135 together with the secondary endoscope 130 by a predetermined amount of rotation (a rotation action 901) which can be less than a single revolution (i.e., less than 360 degrees) to only change the orientation of the distal end 139 of the secondary endoscope 130.

More specifically, when using a secondary endoscope with a rotatable imaging probe, the hollow-shaft motor 231 would normally rotate the probe 220 together with the drive cable 216 in order to scan the sample with the illumination line 140 (refer to FIGS. 1, 3A, and 3B). In addition, it would be advantageous to actively control the orientation of the distal end 139 of the secondary endoscope 130 to minimize the likelihood of navigation errors and to improve patient safety. To that end, for example, the additional rotation mechanism or second motor 290 (shown in FIG. 9) can be configured to selectively engage only with the guide 135 and rotate the guide 135 to place the distal end 139 of the secondary endoscope 130 inside or outside of the field of view 152 of the main endoscope, as shown in FIG. 10A and FIG. 10B.

As shown in FIG. 3A, the probe 220 is arranged inside the drive cable 216, and the hollow-shaft motor 231 normally rotates the drive cable 216 together with the illumination optics of probe 220 in a rotation direction R. A rotation detection unit including, for example, a rotating disc 232 and an encoder module 233 is provided to obtain rotation information of the drive cable 216 with respect to the guide 135. The rotating disc 232 is fixedly attached to the drive cable 216, so that the encoder module 233 can obtain the rotation information of the drive cable 216. The rotation information obtained by encoder module 233 can include the rotation speed, rotation direction (clockwise or counter-clockwise) and/or rotation position (angular position) of the drive cable 216. The rotation information is sent from the encoder module 233 to console 110. At the console 110, the CPU 261 (FIG. 2) uses the rotation information provided by the rotation detection unit and the spectral information obtained from the collected light for the image reconstruction process to form and output a reconstructed image of the arear of interest. The same type of control (i.e., the same rotating disc 232 and encoder module 233) can be used to control the orientation of the distal end 139 of the secondary endoscope to place the secondary endoscope 130 at an orientation that the user prefers. That is, according to the embodiment shown in FIG. 10A and FIG. 10B it is possible to actively place the distal end 139 of the secondary endoscope inside or outside of the field of view 152 of the main endoscope 150, by rotating the endoscope guide 135 and tracking the rotation thereof with the encoder module 233. Alternatively, other position sensor, such as a Hall-effect sensor can be used to sense the rotation of the secondary endoscope 130 with respect to the main endoscope 150 or vice versa.

FIG. 10A illustrates an example of the endoscope system 100 used in the navigation mode where the main endoscope 150 is assembled (joined) together with the secondary endoscope 130 via the mechanical joint 160 prior to being inserted into a lumen. In this configuration, the main endoscope 150 and the secondary endoscope 130 are joined together such that the first endoscope probe is arranged to take forward-viewing live images from a field of view 152 that includes the distal end 139 of the second endoscope probe when the second endoscope probe is moved linearly for navigating towards a specific location of interest. As shown in FIG. 1A, in the navigation mode, the secondary endoscope 130 may be navigated through the lumen without emitting any illumination light 140. As noted above, this arrangement is advantageous because the distal end 139 of the secondary endoscope can be continuously monitored by a live view image of the main endoscope during insertion into a bodily lumen. This procedure can occur during linear movement of insertion into a bodily lumen prior to using the secondary endoscope to obtain side-view images of tangential areas of the bodily lumen.

FIG. 10B shows an example embodiment of the endoscope system 100 used in the imaging mode where the secondary endoscope 130 (or more precisely, the endoscope guide 135) is actively rotated so that the distal end 139 is moved out of the field of view 152 of the main endoscope 150 for obtaining an image of an area tangential or lateral to the field of view 152. Here, a rotation action 901 of the endoscope guide 135 indicates a pivoting movement of the bent portion of the endoscope 130 while the straight portion of the endoscope 130 remains attached to the main endoscope 150. This pivoting movement for rotation action 901 can be achieved, for example, when the two endoscopes are joined together by a mechanical joint 161a or 161b as shown in FIG. 6A or a cylindrical sleeve as shown in FIG. 7. In this case too, a graphic object such as that shown in any of FIG. 4B, FIG. 5B, or FIG. 6B can be used to inform the user of the exact position and orientation of the tip of the secondary endoscope 130 with respect to field-of-view of the main endoscope 150.

In other words, while one mode of operation of the dual endoscope system 100 would be to have the main and secondary scopes move/rotate together as a unit, the dual endoscope system 100 can also be operated in a manner that the main endoscope 150 and the secondary endoscope 130 can move and/or rotate independent from each other as shown in FIG. 10A and FIG. 10B. Once the secondary endoscope 130 is safely navigated together with main endoscope 150 through the bodily lumen to a target area of interest (as shown in FIG. 10A), the secondary endoscope 130 can be independently rotated or guided to image areas tangential or lateral to the field of view.

### <Tracking Procedure>

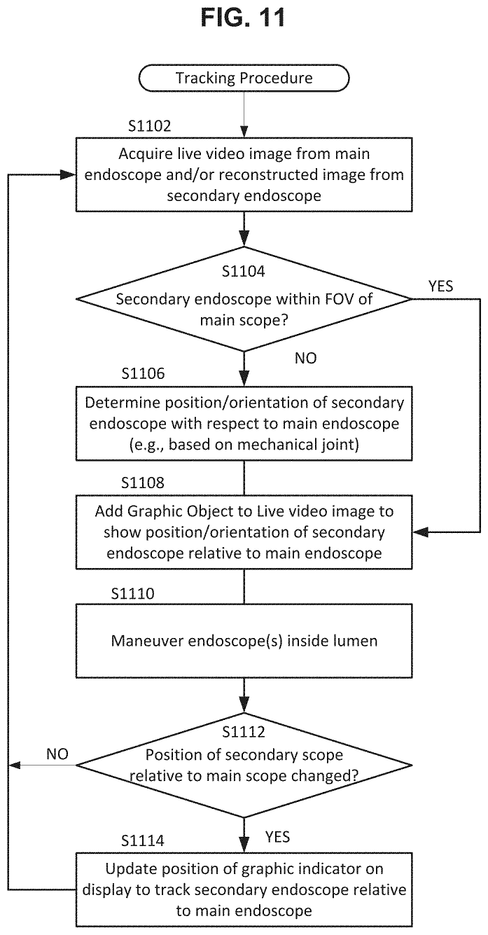

FIG. 11 shows a flowchart of an example tracking procedure (tracking algorithm) for the dual endoscope system 100. The operation of the tracking procedure is described in connection with the dual endoscope system 100 shown in FIG. 1 and the various embodiments of the mechanical joint 160. Referring to FIG. 11, the tracking procedure includes a step S1102 which assumes the main endoscope 150 and secondary endoscope 130 are joined together as unit and inserted into a lumen or cavity of patient. Once inserted into a lumen or cavity, at step S1102, the system 100 acquires a live video image from the main endoscope 150, and it may also obtain an image from the secondary endoscope 130. At step S1104, a determination is made as to whether the distal end of the secondary endoscope 130 is present within the field of view (FOV) of the main endoscope. The determination at step S1104 can be made by the user observing the acquired live video image in the display device 115, and providing a manual input for the flow process executed by the system 100. Alternatively, the determination at step S1104 can be made by image analysis (software analysis) of the live video image to determine if an image of the distal end of secondary endoscope 130 is detected within one or more frames of the video stream.

In the case where a positive determination is made (YES in S1104) asserting that the secondary endoscope is within the FOV of the main endoscope, the process advances to step S1108 where the system 100 adds a graphic object to the live video image shown in the display device. For example, the display device adds a graphic object 505 as shown in FIG. 5B. In the case where a negative determination is made (NO in S1104) indicating that the secondary endoscope is not observed within the FOV of the main endoscope, the process advances to step S1106 where the system 100 actively determines the position and/or orientation of the secondary endoscope with respect to the main endoscope. As previously described, the secondary endoscope 130 can be attached to the main endoscope 150 with an orientation pointing away from the main endoscope (e.g., as shown in FIG. 4A). In this case, the distal end of the secondary endoscope 130 will not be seen in the live video image of main endoscope 150. However, the position of the secondary endoscope 130 with respect to the main endoscope 150 can be known before the two endoscopes are inserted into the lumen. Alternatively, the position and orientation of the secondary endoscope 130 can be sensed or determined, e.g., by image guidance. Therefore, at step S1106, the system 100 may receive the known position of the secondary endoscope 130, and then at step S1108 the system will add a graphic object to the live video image shown in the display device. For example, at step S1108, the display device adds a graphic object 405 as shown in FIG. 4B.

At step S1110, while the joined main and secondary endoscopes advance through the lumen, or when the main or secondary endoscopes are maneuvered inside the lumen at a desired target location, the system 100 continues to track the relative position and orientation of the secondary endoscope with respect to the main endoscope. That is, at step S1112, the system makes a determination as to whether the position of the secondary endoscope 130 relative to the main endoscope 150 has changed. In the case where the relative position of the main and secondary endoscope has not changed (NO at S112), the system continues to acquire live video images (returns to S1102) and repeats the process of displaying the graphic object together with the live video images. On the other hand, in the case where the relative position of the secondary endoscope relative to the main endoscope has changed (YES at S112), the flow advances to S114 where the system 100 updates the position of the graphic object with respect to the live image on the display 115. After the position of the graphic object is updated, the system continues to acquire live video images (returns to S1102) and repeats the process of displaying the graphic object together with the live video images until the tracking process is terminated at the user discretion. In this manner, the system 100 can be configured to change or update the graphic object in real time to track the relative position and orientation of the secondary endoscope 130 with respect to the main endoscope 150.

### <Exemplary Application>

According to one or more of the embodiments described herein, the dual endoscope system 100 can be implemented as a nasal endoscope. Nasal endoscopy allows a detailed examination of the nasal and sinus cavities of a patient. Nasal endoscopy is typically performed by an Otolaryngologist (Ear Nose Throat doctor) using either a zero degree or an angled nasal endoscope. Nasal endoscopy is a method of evaluating medical problems such as nasal stuffiness and obstruction, sinusitis, nasal polyps, nasal tumors, and epistaxis (nose bleeds). Typically, nasal endoscopy is performed with a zero degree endoscope using the “three pass” technique, visualizing three main areas in the nasal and sinus cavities. The zero degree nasal endoscope allows a straight view from the tip of the instrument into the nose. In the first pass the nasal floor and the back of the nose (nasopharynx) are viewed. The endoscope is then brought out and turned upwards and sideways in order to view the drainage areas of the nasal sinuses (middle and superior meati and the spheno-ethmoidal recess), in a second pass. In the third pass, the endoscope is used to view the roof of the nose and the area of the olfactory cleft (smell region). The “angled” (30/45/70 degree) endoscopes, in which the view is at an angle from the tip of the endoscope, provide an “around the corner” view, deep into the sinus cavities. However, the angled endoscope does not provide direct straight view into the nasal passages, so there is a possibility for navigation errors or patient injury.

Therefore, with either modality (i.e., with zero degrees or angled endoscopes), in order to minimize patient discomfort, just before nasal endoscopy the nose will be sprayed with a nasal decongestant and a local anesthetic. The nasal decongestant is used to reduce the swelling in the nasal membranes to permit an easy passage of the endoscope; and the local anesthetic temporarily numbs the nose of a patient, and helps decrease the chances of sneezing from patient's sensitivity to foreign objects. Nevertheless, some patients may experience discomfort if the nasal cavity is unusually narrow or the nasal lining is swollen. Moreover, potential complications such as mucosal trauma and bleeding may occur, particularly in susceptible patients with increased risk of bleeding.

The dual endoscope system 100 disclosed herein improves on the above-described conventional “three pass” technique and avoids (or at least) significantly reduces navigation error and patient injury because the main endoscope and the secondary endoscope are joined together along a length of the first and second tubular shafts and are configured to be simultaneously inserted into a lumen, wherein the main endoscope is arranged to acquire a first image of a field of view from inside the lumen, and the secondary endoscope is arranged to acquire a second image of an area which is tangential or lateral to the field of view, and wherein the display device displays the first image acquired by main endoscope and a graphic object depicting a position and orientation of the secondary endoscope with respect to the main endoscope. Moreover, the graphic object updates in real time to track movement of the secondary endoscope with respect to the main endoscope.

The dual endoscopy system 100 described herein offers the following advantages, among others: (a) avoidance of reduction of injury to the patient, in particular to the inner surface of a bodily lumen, due to the possibility of simultaneous optical monitoring via the acquisition of images with both the main endoscope and secondary endoscope; (b) in contrast to the multi-pass technique for nasal endoscopy, the nasal insertion of the endoscope does not require multiple insertions because the main endoscope can image the field of view directly in front of the main endoscope, while the secondary endoscope can image areas of lumen which are tangential or lateral to the field of view; (c) in contrast to conventional dual endoscopy operation, which requires two operators, the dual endoscope system described herein requires a single operator; this naturally reduces operation costs; (d) the graphic object updates in real time to track movement of the secondary endoscope with respect to the main endoscope.

In referring to the description, specific details are set forth in order to provide a thorough understanding of the examples disclosed. In other instances, well-known methods, procedures, components and circuits have not been described in detail as not to unnecessarily lengthen the present disclosure. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

While the present disclosure has been described with reference to exemplary embodiments, it is to be understood that the present disclosure is not limited to the disclosed exemplary embodiments. The scope of the following claims is to be accorded the broadest interpretation so as to encompass all such modifications and equivalent structures and functions.

1. An endoscope system, comprising:
a main endoscope having a first tubular shaft which is rigid and substantially straight extending from a proximal end to a distal end;
a secondary endoscope having a second tubular shaft which has a straight portion and a bent portion at the distal end thereof; and
a display device configured to display an image received from the main endoscope and/or the secondary endoscope,
wherein the main endoscope and the secondary endoscope are joined together along a length of the first and second tubular shafts and are configured to be simultaneously inserted into a lumen,
wherein the main endoscope is arranged to acquire a first image of a field of view from inside the lumen, and the secondary endoscope is arranged to acquire a second image of an area of the lumen which is at an angle to the field of view of the main endoscope, and
wherein the display device displays the first image acquired by main endoscope and a graphic object depicting a position and/or orientation of the secondary endoscope with respect to the main endoscope.

2. The endoscope system according to claim 1,
wherein the first image is a live video image of the field of view from inside the lumen, and the second image is a reconstructed two dimensional image of an area which is tangential or lateral to the field of view, and
wherein the display device displays the live video image simultaneously with the graphic object.

3. The endoscope system according to claim 2,
wherein the bent portion of the secondary endoscope is arranged to point away from the main endoscope, and
wherein the display device displays the live video image in a central area of a screen, and displays the graphic object outside the live video image so as to indicate the orientation of the secondary endoscope with respect to the field of view of the main endoscope.

4. The endoscope system according to claim 2,
wherein the bent portion of the secondary endoscope is arranged to point away from the main endoscope such that the secondary endoscope is used to view portions of the lumen which are tangential to the field of view of the main endoscope, and
wherein the display device displays the live video image and the graphic object superposed on a part of the live video image.

5. The endoscope system according to claim 2,
wherein the bent portion of the secondary endoscope is arranged to point towards the main endoscope such that the first image includes an image of the distal end of the secondary endoscope, and
wherein the display device displays the live video image with the image the distal end of the secondary endoscope and without the graphic object.

6. The endoscope system according to claim 2,
wherein the bent portion of the secondary endoscope is arranged to point towards the main endoscope such that the first image includes an image of the distal end of the secondary endoscope, and
wherein the display device displays the live video image with the graphic object superposed on part of the live video image.

7. The endoscope system according to claim 2,
wherein the graphic object updates in real time to track movement of the secondary endoscope with respect to the main endoscope.

8. The endoscope system according to claim 2,
wherein the main endoscope comprises an imaging chip disposed at the distal end of the first tubular shaft,
wherein the imaging chip is positioned such that the imaging chip images the field-of-view distal to the distal end of the main endoscope.

9. The endoscope system according to claim 2,
wherein the secondary endoscope comprises a spectrally encoded endoscopy (SEE) probe, and
wherein the SEE probe is configured to image an area tangential or lateral to the field of view with a spectrally-encoded illumination light.

10. The endoscope system according to claim 1, further comprising a mechanical joint configured to joint together the main endoscope and the secondary endoscope in a permanent manner.

11. The endoscope system according to claim 10, wherein the main endoscope and the secondary endoscope are configured to move linearly inside the lumen and rotate inside the lumen as a unit without changing their relative position or orientation thereof.

12. The endoscope system according to claim 1, further comprising a mechanical joint configured to joint together the main endoscope and the secondary endoscope in a temporary manner.

13. The endoscope system according to claim 12, wherein the main endoscope and the secondary endoscope are configured to move linearly inside the lumen together as unit, and rotate inside the lumen independent from each other, such that the secondary endoscope changes at least its orientation with respect to the main endoscope.

14. A dual endoscope system for performing a medical procedure, comprising:
a first endoscope probe having opposite proximal and distal ends, wherein at least a distal portion of the first endoscope probe is rigid and substantially straight and configured to be inserted into a bodily lumen for forward-view imaging;
a second endoscope probe having opposite proximal and distal ends and configured to be inserted into the bodily lumen for side-view imaging, wherein at least the distal end of the second endoscope probe is bent or bendable at an angle with respect to a proximal portion thereof; and
a display device configured to display an image received from the first endoscope probe and/or the second endoscope probe,
wherein the first endoscope probe and the second endoscope probe are joined together substantially parallel to each other such that the first endoscope probe is arranged to take a forward-view image of a field of view that includes the distal end of the second endoscope probe when the second endoscope probe is navigated through the bodily lumen, and
wherein the display device displays the forward-view images acquired by the first endoscope probe and a graphic object depicting a position and orientation of the second endoscope probe with respect to the first endoscope.

15. The endoscope system according to claim 14, wherein the main endoscope is a zero-degrees endoscope, and the secondary endoscope is a pre-curved non-zero-degree endoscope.

16. The endoscope system according to claim 14, wherein the secondary endoscope is a spectrally encoded endoscopy (SEE) device, and wherein the distal end of the SEE device is configured for forward-view imaging.

17. The endoscope system according to claim 14, further comprising a mechanical joint configured to joint together the first endoscope probe and the second endoscope probe in a permanent manner.

18. The endoscope system according to claim 14, further comprising a mechanical joint configured to joint together the first endoscope probe and the second endoscope probe in a temporary manner.

19. The endoscope system according to claim 14, further comprising:
a processor configured to obtain a live image from the first endoscope probe and a processed endoscopic image from the second endoscope probe; and
a display device configured to display the live image and the processed endoscopic image.

20. A method, comprising:
joining together a main endoscope and a secondary endoscope, wherein the main endoscope having a first tubular shaft which is rigid and substantially straight extending from a proximal end to a distal end; and the secondary endoscope having a second tubular shaft which has a straight portion and a bent portion at the distal end thereof; and
displaying, in a display device, an image received from the main endoscope and/or the secondary endoscope,
wherein the main endoscope and the secondary endoscope are joined together along a length of the first and second tubular shafts and are configured to be simultaneously inserted into a lumen,
wherein the main endoscope is arranged to acquire a first image of a field of view from inside the lumen, and the secondary endoscope is arranged to acquire a second image of an area which is tangential or lateral to the field of view,
wherein the display device displays the first image acquired by main endoscope and a graphic object depicting a position and orientation of the secondary endoscope with respect to the main endoscope,
wherein the first image is a live video image of the field of view from inside the lumen, and the second image is a reconstructed two dimensional image of an area which is tangential or lateral to the field of view, and
wherein the display device displays the live video image simultaneously with the graphic object.
